(19) United States
(12) Patent Application Publication
(10) Pub. No.: US 2006/0122656 A1
(43) Pub. Date: Jun. 8, 2006

(54) Power supply for medical apparatus

(76) Inventor: Ting-Peng Lai, Chung-Ho City (TW)

Correspondence Address: HDSL, 4331 STEVENS BATTLE LANE, FAIRFAX, VA, 22033, US

(21) Appl. No.: 11/000,955

(22) Filed: Dec. 2, 2004

(51) Int. Cl. A61N 1/378

(52) U.S. Cl. 607/34

(57) ABSTRACT

A power supply for a medical apparatus is utilized to provide power for the medical apparatus. The power supply includes a second connecting portion formed on a face connected corresponding to a first connecting portion formed on a face of the medical apparatus. A conducting wire is electrical connected between and electrical conducting with the power supply and the medical apparatus. Therefore, the power supply can be a secondary power supplier for the medical apparatus such that the medical apparatus has enough power for operation.

### BACKGROUND OF THE INVENTION

The present invention relates in general to a power supply for a medical apparatus, and more particularly, to a power supply for a medical apparatus which is utilized as a second power supplier for supplying enough power to a medical apparatus.

With reference to FIG. 1, a conventional medical apparatus 3 comprises a battery container 31 with a cover 32. The battery container 31 contains a plurality of batteries 33 for supplying power to the medical apparatus 3. Although the battery 33 can supply power for the medical apparatus 3, it may be not enough for supplying the medical apparatus 3 when the medical apparatus 3 is used for a long time.

Therefore, please referring to FIG. 2, the conventional medical apparatus 3 can further comprise a connecting hole 34 for receiving an adapter 4 plugging to a socket to continuously supply power for the medical apparatus 3. However, a conducting wire 41 connected between the medical apparatus 3 and the adapter 4 will limit the using of the adapter 4. The medical apparatus 3 must locate near the socket which causes inconvenience.

### BRIEF SUMMARY OF THE INVENTION

The present invention provides an auxiliary power supply for a medical apparatus. The power supply comprises a second connecting portion formed on a face thereof to connect to a corresponding first connecting portion formed on a face of the medical apparatus. A conducting wire is electrically connected between and conducting with the power supply and the medical apparatus. Therefore, the power supply can be a secondary power supplier for the medical apparatus such that the medical apparatus has enough power for operation.

The objectives of the present invention will become obvious to those of ordinary skill in the art after reading the following detailed description of preferred embodiments.

It is to be understood that both the foregoing general description and the following detailed description are exemplary, and are intended to provide further explanation of the invention as claimed.

### BRIEF DESCRIPTION OF THE DRAWINGS

These as well as other features of the present invention will become more apparent upon reference to the drawings therein:

FIG. 1 is an exploded perspective view of a conventional medical apparatus.

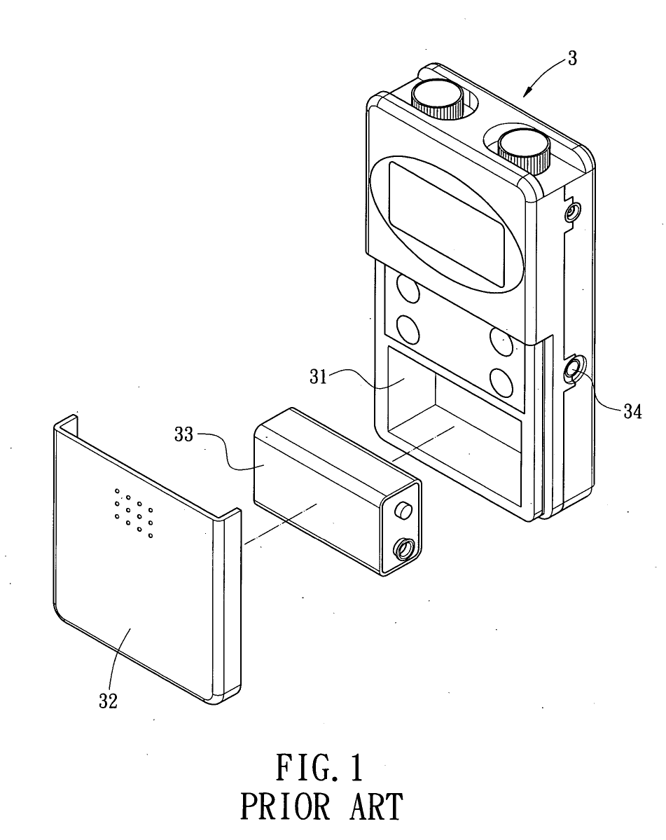

FIG. 2 is a perspective view of the conventional medical apparatus in FIG. 1 connecting with an adapter.

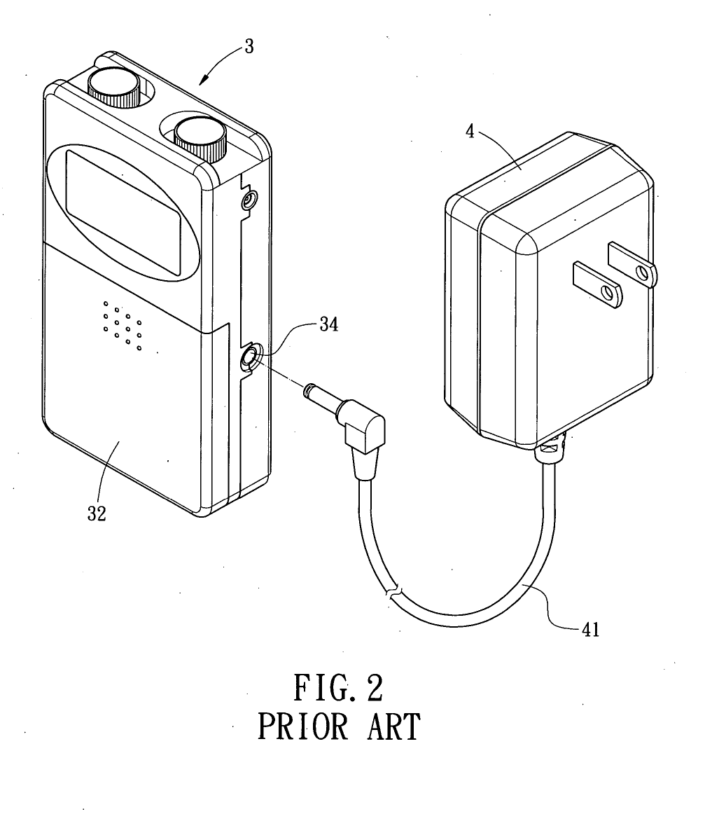

FIG. 3 is an exploded perspective view of a power supply for a medical apparatus in accordance with the present invention.

FIG. 4 is a perspective view of the power supply for the medical apparatus in FIG. 3.

FIG. 5 is another perspective view of the power supply for the medical apparatus in FIG. 3.

FIG. 6 is a side view of the power supply for the medical apparatus in accordance with the present invention.

FIG. 7 is a perspective view of another embodiment of a power supply for a medical apparatus in accordance with the present invention.

### DETAILED DESCRIPTION OF THE INVENTION

Reference will now be made in detail to the preferred embodiments of the present invention, examples of which are illustrated in the accompanying drawings.

Wherever possible, the same reference numbers are used in the drawings and the description to refer to the same or like parts.

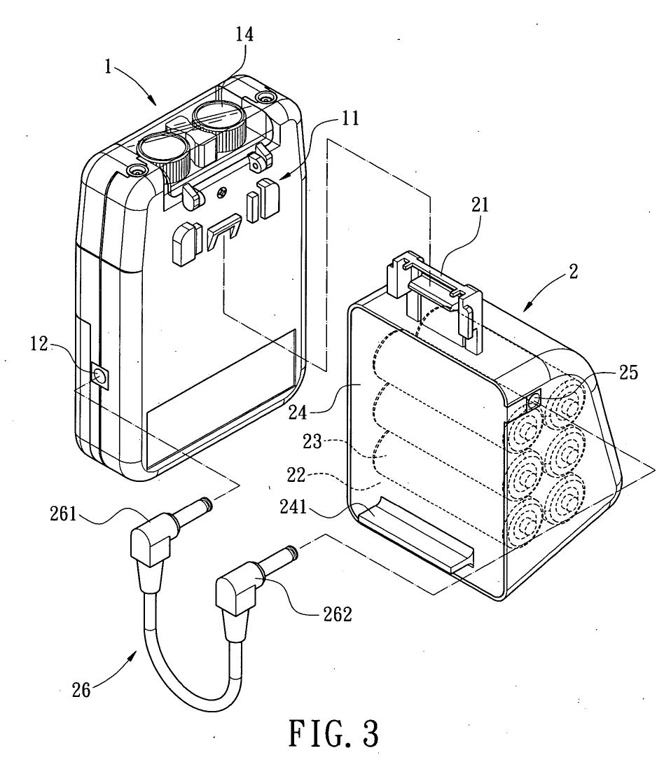

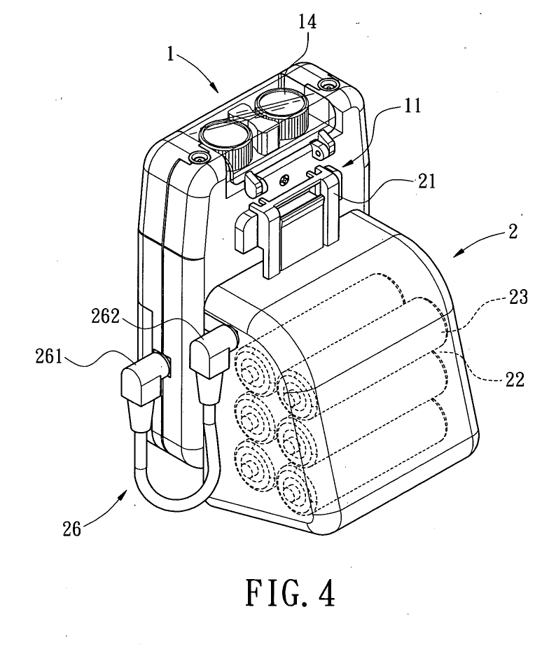

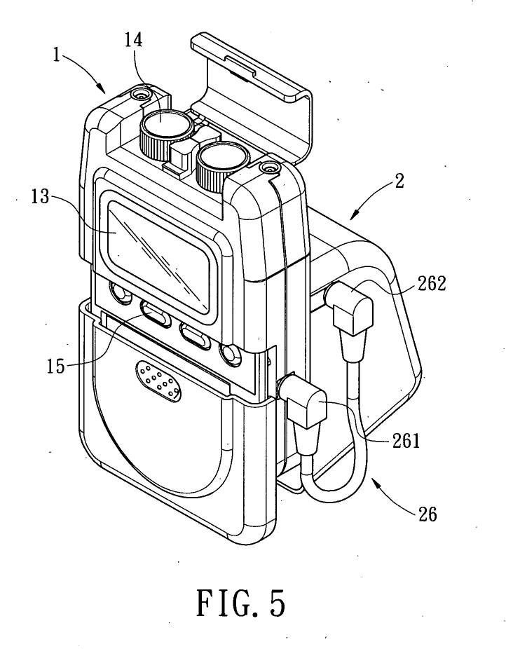

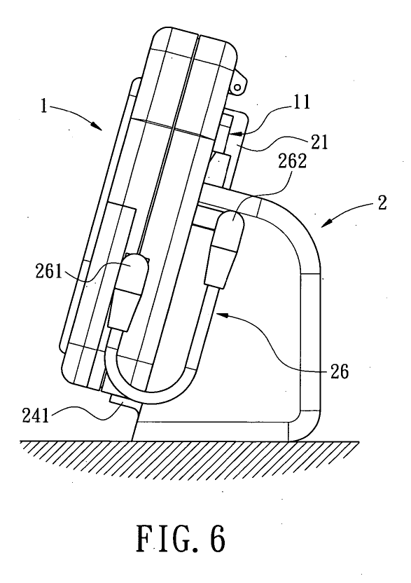

Referring to FIGS. 3 to 6, a second power supply 2 for a medical apparatus 1 in accordance with the present invention is utilized to supply power for the medical apparatus 1 which may contain a traditional battery power supplier and whereby the medical apparatus can maintain enough power.

The medical apparatus 1 can be an interferential therapy unit or any other different therapy unit, which comprises a first connecting portion 11 formed on a face thereof, a first connecting hole 12 formed on one side thereof for connecting with the second power supply 2 or an adapter (not shown) as in conventional use, a display unit 13, a plurality of buttons 14 and a plurality of controlling keys 15.

The power supply 2 comprises a second connecting portion 21 formed on a face thereof for connecting with the first connecting portion 11 of the medical apparatus 1, a battery container 22 for placing at least one battery 23 and a second connecting hole 25 formed in a suitable side thereof. The connecting mechanism of the first connecting portion 11 and the second portion 21 can be an engagement of a guiding slot and a sliding rail to secure the connection of power supply 2 to the medical apparatus 1. The battery container 22 comprises a cover 24 covered thereon and a supporting plate 241 extending from the bottom of the cover 24 incorporated with connecting mechanism so that the power supply 2 of the present invention can be more securely to be mounted to the medical apparatus 1. A conducting wire 26 with two electrical terminals 261, 262 for inserting into the first connecting hole 12 and the second connecting hole 25 respectively connects between the medical apparatus 1 and the power supply 2 for supplying power to the medical apparatus 1.

In use, users can utilize the original battery power supplier set on the medical apparatus 1 and the adapter connected to the first connecting hole 12 for supplying power to the medical apparatus 1 as before. However, users can further utilize the power supply 2 of the present invention as another power supplier so as to place at least one battery 23 in the battery container 22 of the power supply 2 and then the cover 24 covered on one face of the battery container 22. The battery 23 may be a general battery or a rechargeable battery.

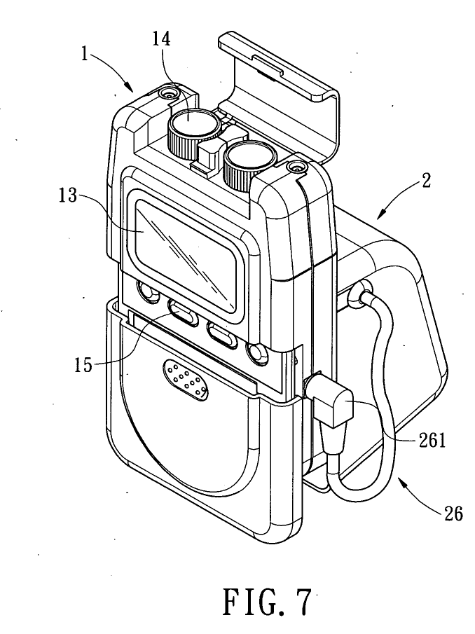

With further reference to FIG. 7, a power supply 2 of another embodiment in accordance with the present invention comprises a different type of conducting wire 26a respect to the foregoing embodiment of the present invention. The conducting wire 26a has two ends. One end directly extends from the power supply. 2 to electrically connect with the battery 23 and the other end has an electrical terminal 261 for inserting into and electrical connecting with the first connecting hole 12 of the medical apparatus 1. Furthermore, the conducting wire 26a can be rolled up and place inside the power supply 2.

While an illustrative and presently preferred embodiment of the invention has been described in detail herein, it is to be understood that the inventive concepts may be otherwise variously embodied and employed and that the appended claims are intended to be construed to include such variations except insofar as limited by the prior art.

What is claimed is:

1. A power supply system, comprising:
a medical apparatus having a primary battery power supplier integrally installed;
a secondary power supplying device mounted to the medical apparatus; and
a conducting wire electrical connected between the secondary power supplying device and the medical apparatus.

2. The power supply system of claim 1, wherein the medical apparatus is an interferential therapy unit.

3. The power supply system of claim 1, wherein the medical apparatus comprises a first connecting portion formed on one face thereof.

4. The power supply system of claim 3, wherein the secondary power supplying device comprises a second connecting portion formed on one face thereof to be engaged with the first connecting portion of the medical apparatus.

5. The power supply system of claim 1, wherein the medical apparatus comprises a first connecting hole formed on one side thereof for receiving an electrical terminal of the conducting wire.

6. The power supply system of claim 1, wherein the secondary power supplying device comprises a second connecting hole formed on one side thereof for receiving an electrical terminal of the conducting wire.

7. The power supply system of claim 1, wherein the secondary power supplying device comprises a battery container for placing at least one battery.

8. The power supply system of claim 7, wherein the battery container comprises a cover covered thereon.

9. The power supply system of claim 8, wherein the battery container comprises a supporting plate extending from the cover for securely mounting the secondary power supplying device to the medical apparatus.

10. The power supply system of claim 1, wherein the conducting wire is directly extended from the secondary power supply with an end formed an electrical terminal for connecting with the medical apparatus.
